(19) United States
(12) Patent Application Publication
(10) Pub. No.: US 2022/0096394 A1
(43) Pub. Date: Mar. 31, 2022

(54) Medical Use of Honokiol

(71) Applicants: Chengdu Jinrui Foundation Biotech Co., Ltd., Chengdu, Sichuan (CN); Beijing Tiantan Hospital, Capital Medical University, Beijing (CN)

(72) Inventors: Wenbin Li, Chengdu, Sichuan (CN); Shenglan Li, Chengdu, Sichuan (CN); Neng Qiu, Chengdu, Sichuan (CN); Haoyu Ye, Chengdu, Sichuan (CN)

(73) Assignees: Chengdu Jinrui Foundation Biotech Co., Ltd., Chengdu, Sichuan (CN); Beijing Tiantan Hospital, Capital Medical University, Beijing (CN)

(21) Appl. No.: 17/276,568

(22) PCT Filed: Dec. 10, 2020
(86) PCT No.: PCT/CN2020/135399

§ 371 Date: Mar. 16, 2021

(30) Foreign Application Priority Data
Sep. 27, 2020 (CN) 202011033093.X

(51) Int. Cl. A61K 31/05; A61P 17/14; A61P 35/00

(52) U.S. Cl. CPC A61K 31/05; A61P 35/00; A61P 17/14

(57) ABSTRACT

The invention involves the pharmaceutical use of honokiol, in particular the use of honokiol in inhibiting medulloblastoma, and it has been experimentally proved that honokiol can inhibit the proliferation and induce cell cycle arrest and apoptosis of medulloblastoma cells; promoting hair growth, and it has been experimentally proved that honokiol can promote hair growth and has no toxic and side effects on liver and kidney; and promoting white hair blackening, and it has been experimentally proved that honokiol can promote white hair blackening and has no toxic side effects on liver and kidney.

### CROSS-REFERENCE TO RELATED APPLICATIONS

This present application is a national phase entry under 35 U.S.C. § 371 of International Application No. PCT/CN2020/135399 filed Dec. 10, 2020, which claims priority from Chinese Patent Application No. 202011033093.X filed Sep. 27, 2020, all of which are incorporated herein by reference in their entirety.

### TECHNICAL FIELD

The invention relates to the medical field, in particular the medical use of honokiol. More particularly, the invention relates to the use of honokiol in inhibiting medulloblastoma, and also the use of honokiol for promoting hair growth and white hair blackening.

### BACKGROUND TECHNOLOGY

Honokiol, chemical name is 3′,5-di-2-propenyl-1,1′-biphenyl-2,4′-diphenol, has the following structural formula:

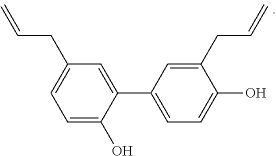 [SMILES: C=CCc1ccc(O)c(-c2ccc(O)c(CC=C)c2)c1]

Honokiol, extracted from the skin of Magnolia Officinalis Rehderet Wilsson, is a small-molecule compound with extensive biological activity. Its main biological activities include anti-inflammatory, antimicrobial, anti-ulcer, antioxidant, anxiolytic, antidepressant, antithrombotic, anti-aging, cholesterol-lowering, etc.

Because honokiol has a wide range of medicinal values, it is necessary to further study the new use of honokiol.

### SUMMARY

After a large number of experiments and studies, the inventor has discovered a new use, i.e., honokiol can be used to inhibit medulloblastoma. Therefore, an aim of the invention is to provide the use of honokiol in the preparation of drugs for inhibiting medulloblastoma.

Based on the use of inhibiting medulloblastoma, wherein the honokiol is prepared as honokiol liposome (Lip-HNK or Lip-HK), preferably honokiol liposome for injection.

Based on the use of inhibiting medulloblastoma, wherein the honokiol inhibits the proliferation of medulloblastoma cells.

Based on the use of inhibiting medulloblastoma, wherein the honokiol induces the apoptosis of medulloblastoma cells. On the one hand, the honokiol induces the apoptosis of medulloblastoma cells through the ROS/ERK/p38MAPK pathway, wherein the honokiol-induced apoptosis of medulloblastoma cells involves ROS generation and the honokiol inhibits the ERK/p38 MAPK signaling pathway by generating excess ROS in medulloblastoma cells. On the other hand, the honokiol induces the apoptosis of medulloblastoma cells through a Caspase (cysteine-containing aspartate proteolytic enzyme)-dependent pathway.

Based on the use of inhibiting medulloblastoma, wherein the honokiol induces the cycle arrest of medulloblastoma cells. Specifically, the honokiol induces G1 cycle arrest of medulloblastoma.

The invention studies the inhibitory effect of Lip-HNK on the proliferation of medulloblastoma cells and its mechanism. Lip-HNK also induces G1 cycle arrest and caspase-dependent apoptosis in medulloblastoma cells, but is not significantly cytotoxic to normal cells. Lip-HNK has been shown to inhibit the growth of tumor cell lines, but the use of Lip-HNK to inhibit medulloblastoma has not been reported, and the molecular mechanism of Lip-HNK on the death of medulloblastoma has not been investigated. This inhibitory effect of Lip-HNK on medulloblastoma may be mediated by induction of intracellular reactive oxygen species (ROS) and loss of mitochondrial membrane potential. At the same time, Lip-HNK inhibits the phosphorylation of ERK and p38 in a dose-dependent manner. More importantly, the effects of Lip-HNK on mitochondrial membrane potential, ROS generation, and phosphorylation of ERK and p38 were found to be significantly reversed by ROS inhibitors, indicating that Lip-HNK affects medulloblastoma cells and ERK/p38 MAPK signaling by generating excess ROS. Therefore, the inventor has elucidated for the first time that medulloblastoma induces the apoptosis of medulloblastoma cells through the ROS/ERK/p38MAPK pathway, providing a basic scientific basis for Lip-HNK becoming a new potential therapy for medulloblastoma.

After a large number of experiments and studies, the inventor also found that honokiol can be used to promote hair growth. Therefore, another aim of the invention is to provide the use of honokiol in the preparation of drugs for promoting hair growth.

Based on the use for promoting hair growth, wherein the honokiol is prepared as honokiol liposome, preferably honokiol liposome for injection.

Based on the use for promoting hair growth, wherein the site of hair growth maybe the head.

The invention studies the effect of honokiol on hair growth, and the experiments prove that honokiol can promote hair growth, in particular, honokiol accelerates hair growth rate, increases hair follicle length, and has no toxic and side effects on liver and kidney.

In addition, after a large number of experiments and studies, the inventor also found that honokiol can be used to promote white hair blackening. Therefore, another aim of the invention is to provide the use of honokiol in the preparation of drugs for promoting white hair blackening.

Based on the use for promoting white hair blackening, wherein the honokiol is prepared as honokiol liposome, preferably honokiol liposome for injection.

The invention studies the effect of honokiol on white hair blackening, and the experiments prove that honokiol can promote white hair blackening, and has no toxic and side effects on liver and kidney. Another aim of the invention is to provide honokiol liposome for inhibiting medulloblastoma/promoting hair growth/promoting white hair blackening.

The honokiol liposome in this invention is honokiol liposome for injection.

The honokiol liposome in this invention may be in the following dosage forms: lyophilized powder formulations, including lyophilized powder for injection, oral lyophilized powder; tablets, including immediate-release tablets and sustained-release tablets; capsules, including hard capsules, soft capsules, sustained-release capsules, and enteric-coated capsules; transdermal preparations, etc.

The honokiol liposome in this invention may be administered by intravenous injection, intramuscular injection, subcutaneous injection, oral administration, ocular administration, pulmonary administration, transdermal administration, nasal administration, etc.

### BRIEF DESCRIPTION OF THE DRAWINGS

FIG. 1A-1C show that Lip-HNK inhibits proliferation of medulloblastoma cells.

FIG. 2A-2E show that Lip-HNK induces cycle arrest of medulloblastoma cells.

FIG. 3A-3D show that Lip-HNK induces apoptotic cell death of medulloblastoma.

FIG. 4A-4F show that Lip-HNK-induced apoptosis of medulloblastoma cells involves ROS generation.

FIG. 5A-5C show that Lip-HNK inhibits the ERK/p38 MAPK signaling pathway by generating excess ROS in medulloblastoma cells.

FIG. 6A-6C show that Lip-HNK induces apoptotic cell death of medulloblastoma cells through a Caspase-dependent pathway.

FIG. 7 shows hair growth at different time points in the normal saline and Lip-HNK groups.

FIG. 8 shows HE slides of hair follicles from mouse dorsal skin tissue at different treatment time points.

FIG. 9 shows that Lip-HNK has no toxic or side effects on the liver and kidney of mice.

FIG. 10 shows that honokiol promotes hair growth as well as white hair blackening.

### DETAILED DESCRIPTION

Experimental examples below are provided to further illustrate the pharmaceutical use of honokiol.

### Experimental Example 1: Honokiol for Inhibition of Medulloblastoma

1. Experimental Materials and Instruments:

Honokiol liposome was provided by Chengdu Jinrui Foundation Biotechnology Co., Ltd.; human medulloblastoma cells (DAOY cells and D283 cells), mouse microglia BV2 cells, and mouse hippocampal neuronal cells HT22 cells were purchased from the Cell Bank of Basic Institute of Peking Union Medical College Hospital.

PBS phosphate-buffered saline, 4% paraformaldehyde, 0.5% crystal violet, methanol, absolute ethanol, CCK-8, reactive oxygen species kit, fetal bovine serum (Gbico), and antibody: caspase-3 (Cat. No.: ab13847), c-caspase-3 (ab32042), ERK (ab17942), p-ERK (ab201015), p38 (ab170099) and secondary antibodies were purchased from Abcam; p-p38 (8690T) and CDK4 (12790) were purchased from Cell Signaling Technology; GAPDH (ZSGB-BIO), Hoechst 33342, propidium iodide PI and apoptosis kit were purchased from BD; JC-10 kit (CA310-100, Solarbio), RIPA lysate (R0020, Solarbio), PVDF membrane (ISEQ00010 Solarbio), 96-well plate, six-well plate and petri dish were purchased from Corning; fluorescence microscopy, plate reader, flow cytometry (BD Biosciences, San Jose, Calif., USA), electrophoresis apparatus, electroporator, and cell incubator were provided by the Basic and Translational Research Laboratories of Beijing Tiantan Hospital.

2. Experimental Methods and Results

The following cellular experiments were performed to analyze the effects and mechanisms of honokiol liposome on medulloblastoma.

Cell Viability Assay

Cell viability was determined by CCK-8. DAOY and D283 cells were seeded in 96-well plates at a rate of 2×10³ cells/well for 24 hours and then treated with different concentrations of Lip HNK for 48 hours. Before the end of treatment, 10 μL of CCK-8 solution was added to each well. After 1-hour incubation, the absorbance was measured at 450 nm.

Clonogenic Assay

In the clonogenic assay, DAOY cells were placed in 6-well plates at a density of 1×10³ cells/well and incubated with different concentrations of Lip HNK at 37° C. Fresh medium was then replaced daily and incubated for 14 days. After fixation in 4% paraformaldehyde and staining with 0.5% crystal violet for 15 minutes, the number of clones was observed.

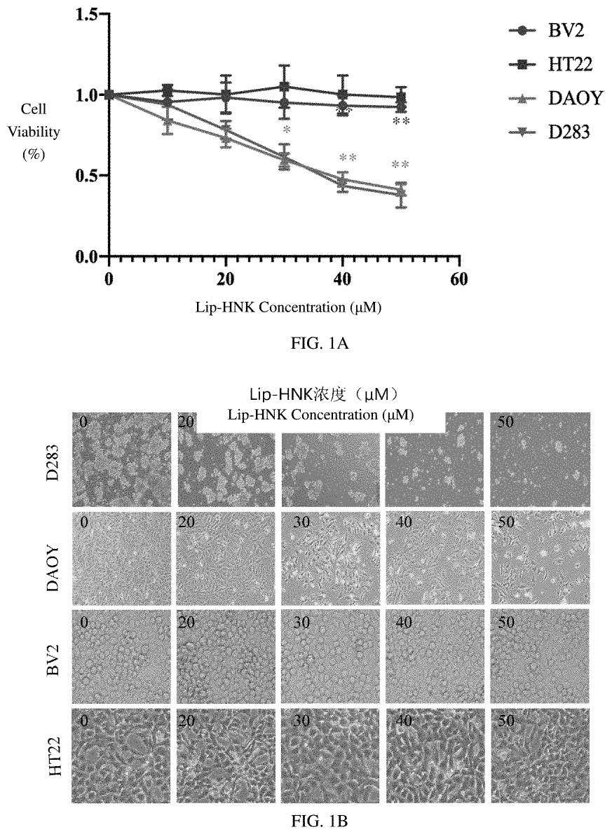

FIG. 1A-1C show that Lip-HNK inhibits proliferation of medulloblastoma cells, wherein FIG. 1A shows the cell viability profile of medulloblastoma cells (DAOY, D283) and normal cells (BV2, HT22) treated with different concentrations of Lip-HNK (0, 20, 30, 40, 50 μM); FIG. 1B shows morphological changes of cells induced by Lip-HNK; FIG. 1C shows representative image of the clonogenic assay of DAOY cells.

Cell Cycle Analysis

DAOY and D283 cells were seeded in 6-well plates at a density of 5×10⁵ cells/well and treated with different concentrations of Lip HNK for 48 hours. Floating and adherent cells were collected and cells fixed in 70% ethanol were placed for at least 24 hours at minus 20 degrees. After all cells were fixed, cycle testing was performed. The specific steps were: take the fixed cells, add 5 mL of cold PBS, and centrifuge at 1500 rpm for 10 minutes. The supernatant was removed, cell pellet left. The cell pellet was resuspended with 2 mL of cold PBS and centrifuged at 1500 rpm for 10 minutes to obtain cell pellet. Finally, the cell pellet was resuspended with 2 mL of cold 2% FBS/PBS and centrifuged at 1500 rpm for 10 minutes to obtain cell pellet. The cells were resuspended with an appropriate amount of PI/RNAase staining solution, and stained at room temperature protected from light for 30 minutes, and then tested.

Hoechst 33342 Staining

For Hoechst 33342 staining, DAOY and D283 cells were pretreated with different concentrations of Lip-HNK (0, 20, 30, and 40 μM) for 48 hours, washed with cold PBS, and fixed in cold methanol. The cells were stained with Hoechst 33342 (1 μg/mL) for 15 minutes and the morphological characteristics of apoptotic cells were observed by fluorescence microscopy.

Determination of Apoptosis by Annexin V and PI Staining

Apoptotic cell death was determined using the Apoptosis Determination Kit (Annexin V-PI: BD Biosciences, San Jose, Calif., USA). 5×10⁵ DAOY and D283 cells were treated with different concentrations of Lip-HNK (0, 20, 30, and 40 μM) for 48 hours. Adherent cells and isolated cells were taken, washed once with PBS and stained with Annexin V-FITC and propidium iodide (PI) at 37° C. for 15 minutes. Apoptosis was determined by flow cytometry.

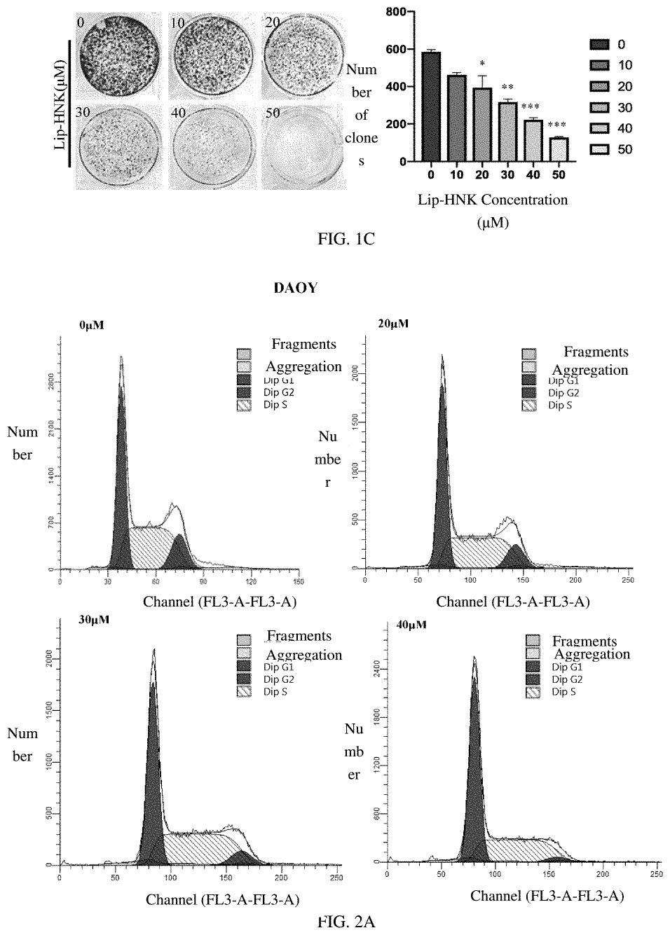

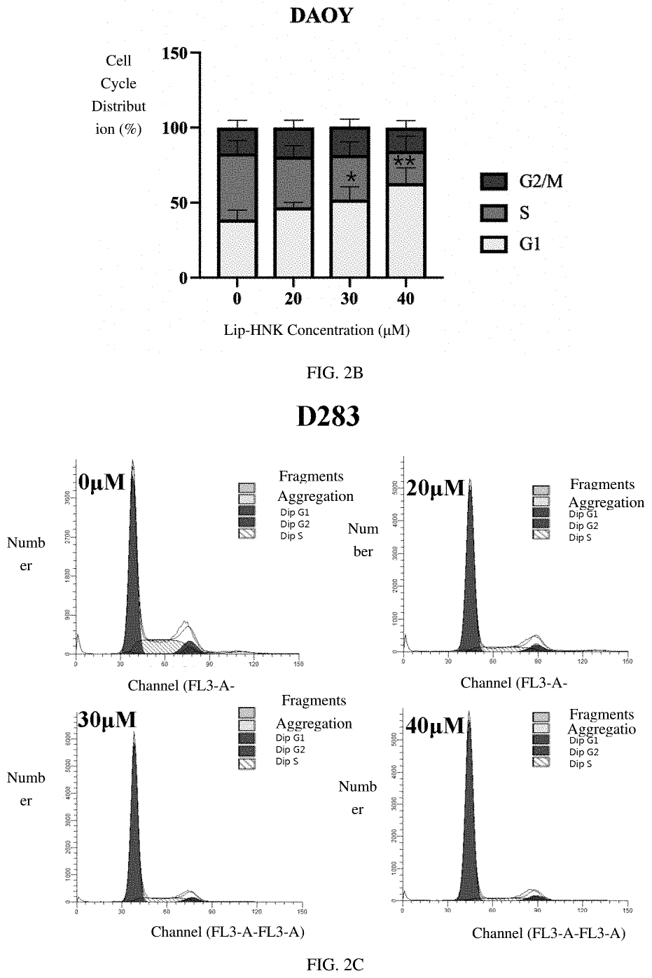

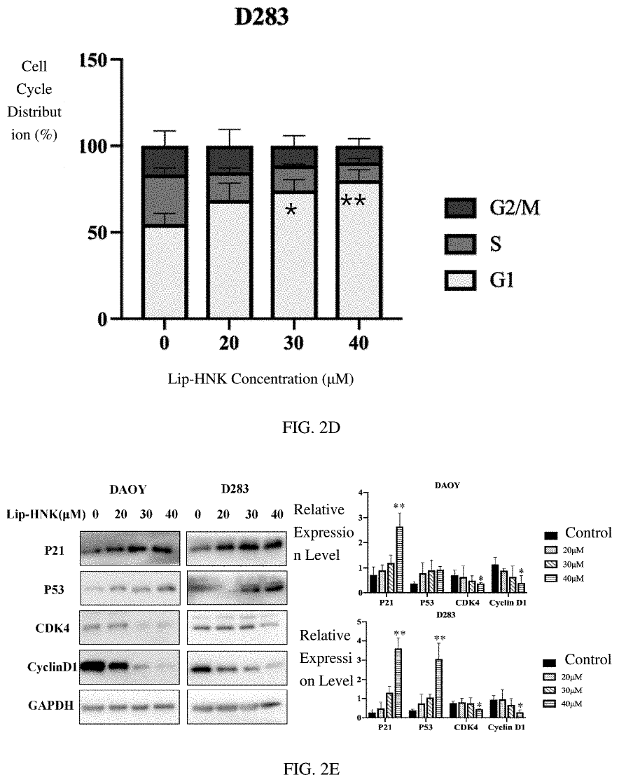

FIG. 2A-2E show that Lip-HNK induces cell cycle arrest of medulloblastoma cells, wherein FIGS. 2A and 2C show the cell cycle distribution of DAOY and D283 cells treated with different concentrations of Lip-HNK; FIGS. 2B and 2D show representative images of cell cycle distribution (%) of DAOY and D283 cells analyzed by flow cytometry; FIG. 2E shows the expression level of P21 protein by western blot after 48 hours of Lip-HNK treatment of DAOY and D283 cells.

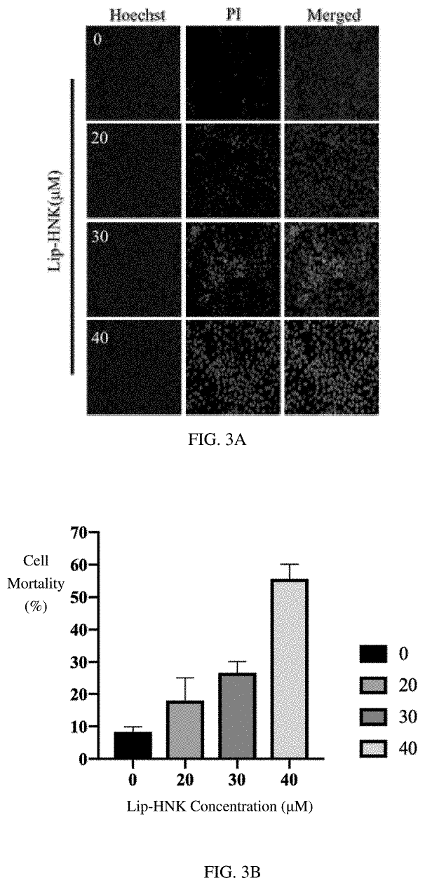

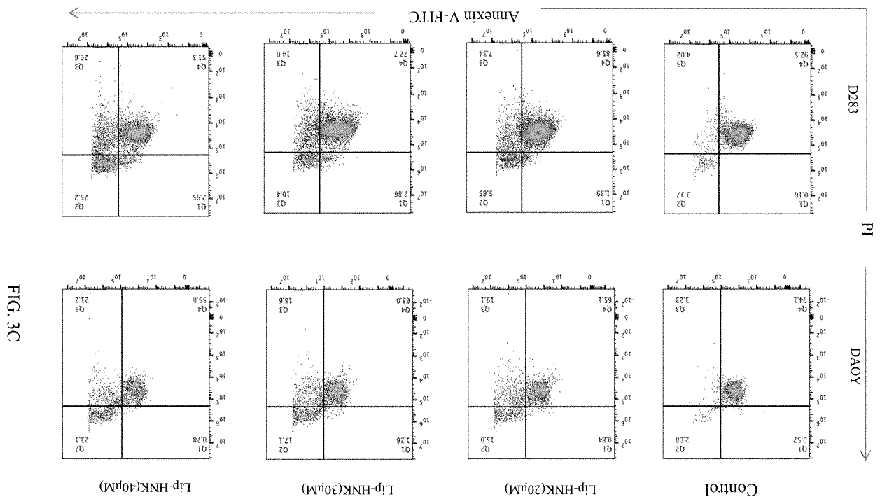

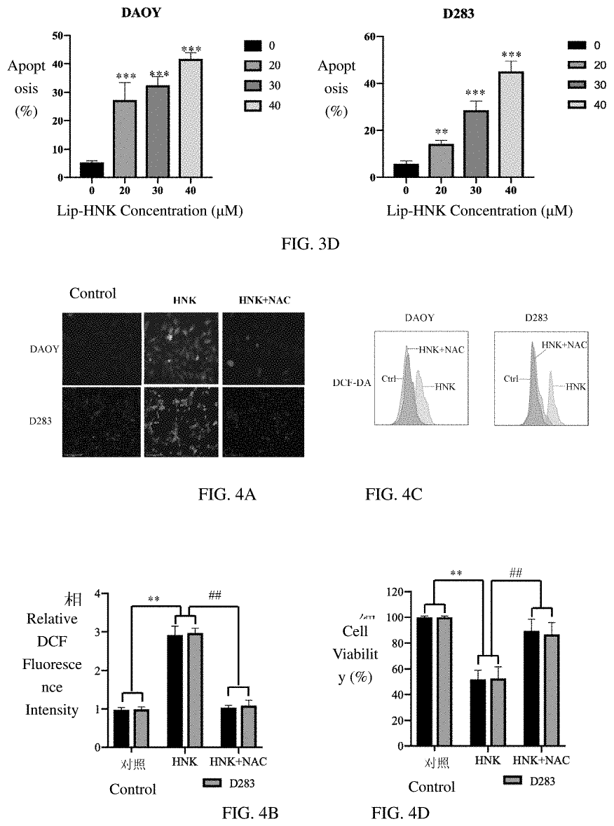

FIG. 3A-3D show that Lip-HNK induces apoptotic cell death of medulloblastoma, wherein FIG. 3A shows analysis of the nuclear structure of DAOY cells by fluorescence microscopy; FIG. 3B shows the ratio of PI staining (red) and Hoechst 33342 staining (blue), which represents cell mortality; FIG. 3C shows apoptotic cell death as determined by Annexin V/PI flow cytometry; FIG. 3D shows the percentage of apoptotic cells.

Intracellular ROS Assay

DAOY and D283 cells were treated with different concentrations of Lip-HNK (0, 20, 30, and 40 μM) for 48 hours, then washed with cold PBS and incubated in 10 μM DCFH-DA for 30 minutes at 37° C. in the dark. DCF fluorescence was measured using a flow cytometer (BD Biosciences, San Jose, Calif., USA) and the data were analyzed using FlowJo10. The fluorescence intensity of intracellular DCF represented ROS levels and was quantified with Image J.

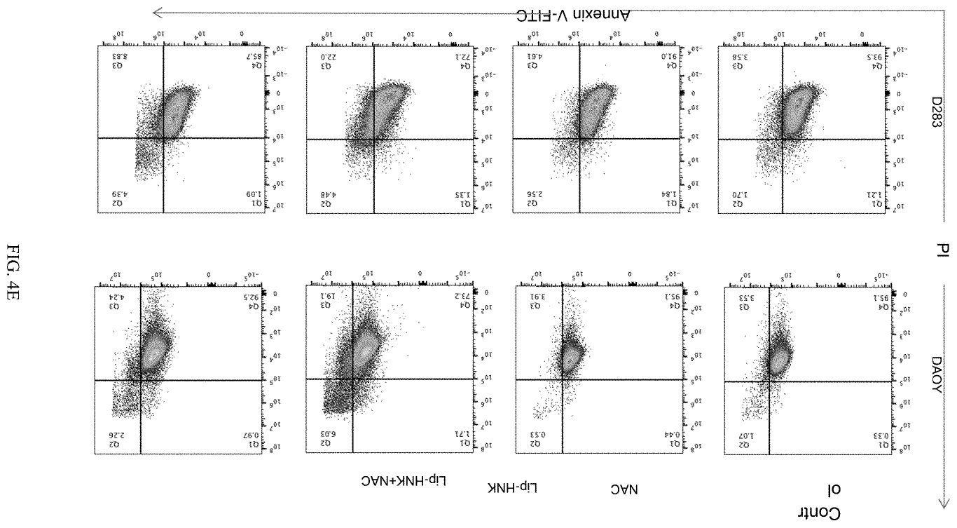

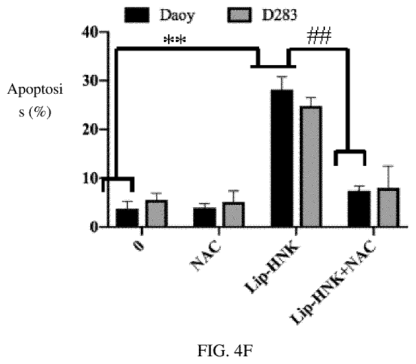

FIG. 4A-4F show that Lip-HNK-induced apoptosis of medulloblastoma cells involves ROS generation, wherein FIG. 4A shows ROS generation in cells treated with Lip-HNK as measured by fluorescence microscopy using DCFH-DA staining; FIG. 4B shows that among three groups (control, Lip-HNK and Lip-HNK+NAC (N-acetyl-L-cysteine)), the relative DCF fluorescence intensity expressed as multiples of the fluorescence intensity of the Lip-HNK and Lip-HNK+NAC groups relative to the fluorescence intensity of the control group; FIG. 4C shows the inhibitory effect of NAC on and honokiol liposome-induced ROS generation as measured by flow cytometry; FIG. 4D shows the percentage of cell viability determined by CCK-8; and FIGS. 4E and 4F show cells stained with Annexin V/PI by flow cytometry.

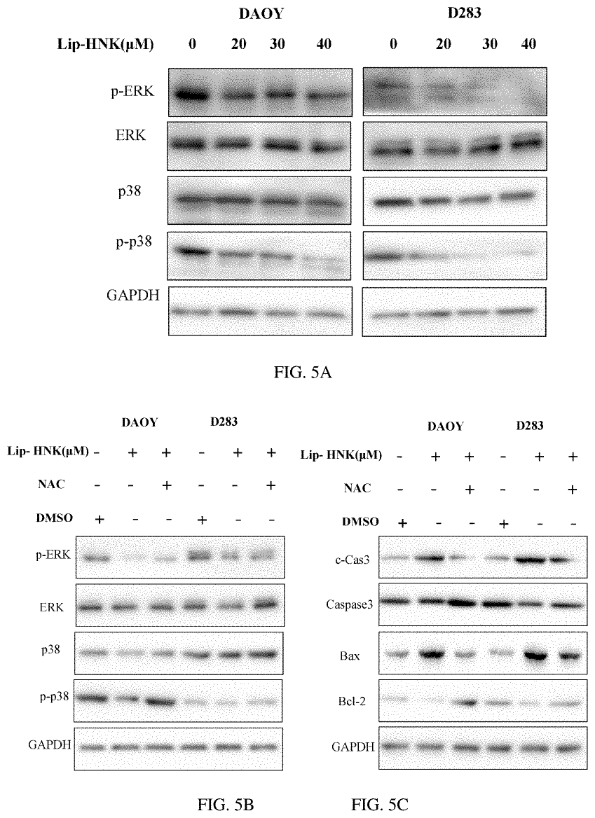

FIG. 5A-5C show that Lip-HNK inhibits the ERK/p38 MAPK signaling pathway by generating excess ROS in medulloblastoma cells, wherein FIG. 5A shows the protein levels of p-ERK, ERK, p-p38, p38 of DAOY and D283 cells after treating with Lip-I-INK; FIG. 5B shows that Lip-HNK acts on medulloblastoma cells via phosphorylation of ERK/p38 MAPK. Medulloblastoma cells were treated with 40 μM Lip-HNK and 5 mM NAC, and then protein levels of p-ERK, ERK, p-p38, p38 were detected by immunoblotting; FIG. 5C shows protein levels of apoptosis-related factors (including cleaved Caspase 3, Caspase 3, Bax, and Bcl-2) detected by immunoblotting.

Mitochondrial Membrane Potential Determination

Mitochondrial membrane potential was measured by JC-10 kit. The DAOY and D283 cells (2×10⁵) were seeded, treated with Lip-HNK for 48 h, then incubated with JC-10 for 30 min at 37° C. and washed twice with PBS. The changes of MMP (mitochondrial membrane potential) were measured by flow cytometry. The positive control cells were treated with CCCP (reactive oxygen species positive control reagent).

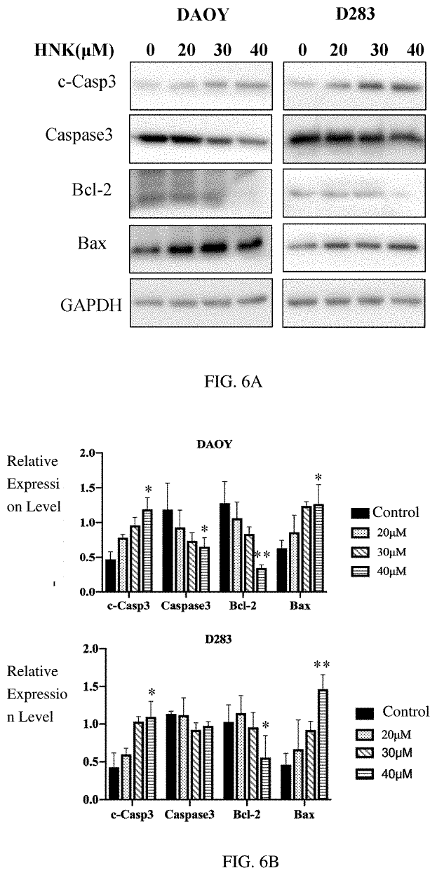

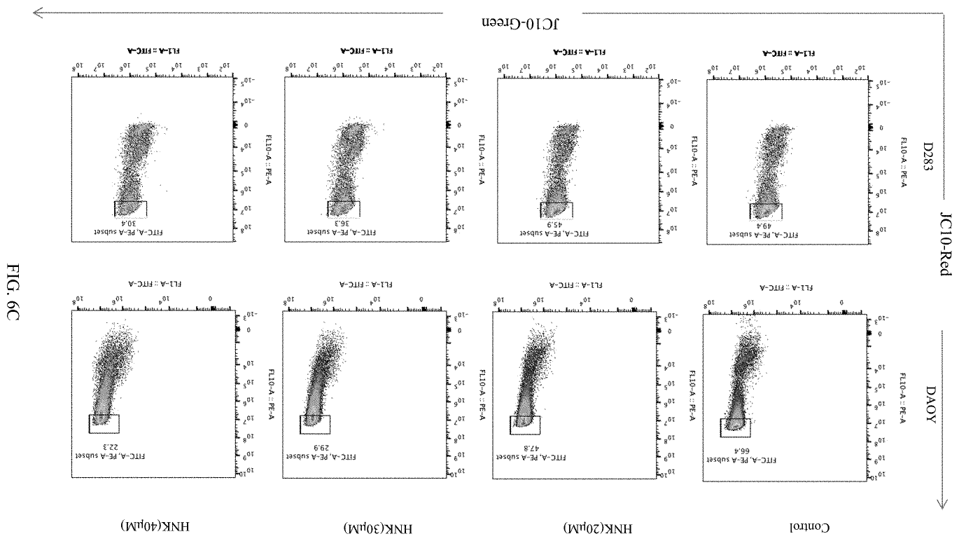

FIG. 6A-6C show that Lip-HNK induces apoptotic cell death of medulloblastoma cells by a Caspase-dependent pathway, wherein FIG. 6A shows expression levels of apoptotic proteins (Bcl-2, Bax, Caspase-3, and cleaved Caspase-3) evaluated by Western blot; FIG. 6B shows data obtained from at least three independent experiments, where values are mean±SD (relative to the control group), *: p<0.05, **: p<0.01; FIG. 6C shows the assessment of MMP using the fluorescent mitochondrial probe JC-10, where the red/green fluorescence intensity was analyzed by flow cytometry.

The above results show that Lip-HNK had inhibitory effect on medulloblastoma cells. On the one hand, Lip-HNK induces medulloblastoma cell apoptosis through the ROS/ERK/p38MAPK pathway; on the other hand, Lip-HNK also induces G1 cycle arrest and caspase-dependent apoptosis in medulloblastoma cells. However, there was no obvious cytotoxicity to normal cells. The inhibitory effect of Lip-HNK on medulloblastoma is mediated by induction of intracellular ROS and loss of mitochondrial membrane potential. At the same time, Lip-HNK inhibits the phosphorylation of ERK and p38 in a dose-dependent manner. The effects of Lip-HNK on mitochondrial membrane potential, ROS generation, and phosphorylation of ERK and p38 can be significantly reversed by ROS inhibitors, indicating that Lip-HNK affects medulloblastoma cells and ERK/p38 MAPK signaling by generating excess ROS, which provides a basic scientific rationale for Lip-HNK becoming a new therapy for medulloblastoma.

### Experimental Example 2: Honokiol for Promoting Hair Growth

1. Experimental Materials and Instruments

Honokiol liposome was provided by Chengdu Jinrui Foundation Biotechnology Co., Ltd.; C57BL/6 mice were purchased from Beijing Charles River Experimental Animal Technology Co., Ltd.; 4% paraformaldehyde (manufacturer: Beyotime; Cat.# P0099), 5% chloral hydrate (Shanghai Yuanmu, R18184), depilating cream, syringe, PBS, distilled water/tap water, xylene, gradient ethanol, neutral gum, paraffin, paraffin embedding machine, microtome, dryer, and microscopes were provided by the Basic and Translational Research Laboratories of Beijing Tiantan Hospital.

2. Experimental Methods and Results

6-week-old C57BL/6 mice weighing 18-20 g were used as experimental animals. After being purchased, the mice were acclimated for three days, and then the mice were depilated on back. The honokiol liposome 20 mg/kg was used for animal experiments. The hair growth of mice in different treatment groups with different number of dosing days (10 days, 14 days, 21 days) was statistically compared using HE staining technique and SPSS software, including the days to skin color change at the dosing site, the days to new hairs growth, the length of hair follicles, etc., to study the effects of honokiol liposome on hair regeneration in mice. The specific experiments are as follows:

1) Hair Growth of Mice at Different Time Points

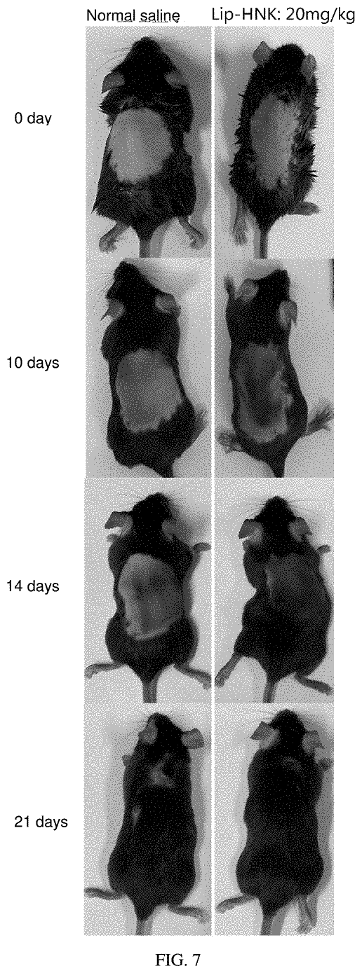

6-week-old C57BL/6 mice were weighed and anesthetized with 5% chloral hydrate, and then depilated with depilating cream on back. Photographs were taken after depilation. Normal saline and honokiol liposome (20 mg/kg) were injected intraperitoneally every day after depilation. Skin and hair growth were observed daily, and hair was measured after growth. Photographs were taken on Day 0, 10, 14, and 21 after depilation. Skin, liver and kidney tissues were taken for paraffin embedding, and tissues were preserved in liquid nitrogen simultaneously. As shown in FIG. 7, Table 1 and Table 2, the time to skin blackening and the time to hair growth were shorter in the honokiol liposome group than those in the normal saline control group.

TABLE 1
Time to Skin Blackening at Dosing Site of Mice after Dosing

| | Normal saline | Honokiol liposome |
|---|---|---|
| Groups | control group (day) | group (day) |
| Time to skin blackening | 7.23 ± 0.45 | 5.19 ± 0.68 |
| after dosing | | |

TABLE 2
Time to New Hair Growth in Mice after Dosing

| | Normal saline | Honokiol liposome |
|---|---|---|
| Groups | control group (day) | group (day) |
| Time to new hair | 12.69 ± 0.49 | 8.19 ± 0.68 |
| growth after dosing | | |

2) HE Slides of Hair Follicles from Mouse Dorsal Skin Tissue at Different Treatment Time Points

Skin tissues from Day 0, 10, 14 and 21 after depilation were taken for paraffin embedding and stained with HE.

Paraffin embedding: fresh skin tissue was taken and unidirectionally cut with a blade into a tissue block of approximately 3-5 mm×3-5 mm×10-20 mm;

Fixation: the tissue block was fixed in 4% paraformaldehyde, with a volume ratio of 1:20 (tissue block: 4% paraformaldehyde); after fixation, the tissue block was washed 3 times with PBS for 5 minutes each time;

Dehydration and clearing: the step should be timed appropriately by different tissues; the basic flow chart is as follows: 75% ethanol-85% ethanol-95% ethanol 1-95% ethanol 2-anhydrous ethanol 1-anhydrous ethanol 2-xylene 1-xylene 2-xylene 3;

Wax immersion: the paraffin wax was melted, and the temperature was maintained at about 57° C.;

Embedding: the tissue block was placed in a mold containing wax solution, and the required tissue section was parallel to the bottom; this step should be as fast as possible as the wax solution is easy to coagulate in a cold environment.

HE staining method and procedure: (1) sections were immersed in xylene for 5-10 min, (2) immersed in xylene for 5-10 min, (3) 100% alcohol for 1 min, (4) 100% alcohol for 1 min, (5) 95% alcohol for 1 min; (6) 95% alcohol for 1 min; (7) 90% alcohol for 1 min; (8) 80% alcohol for 1 min; (9) washed with tap water for 1 min; (10) immersed in hematoxylin for 10-15 min to dye; (11) washed with tap water for 30 sec-1 min; (12) differentiated with 1% hydrochloric alcohol for 30 sec; (13) washed with running water for more than 15 min; (14) stained with 1% eosin alcohol for 3-5 min; (15) differentiated with 90% or 95% alcohol for 30 sec; (16) 95% alcohol for 30 sec-1 min; (17) 95% alcohol for 30 sec-1 min; (18) 95% alcohol for 30 sec-1 min; (19) 100% alcohol for 1 min; (20) 100% alcohol for 1-2 min; (21) xylene carbonate for 1 min; (22) xylene for 1-2 min; (23) xylene for 1-2 min; (24) xylene for 1-2 min; and (25) sealed with neutral gum.

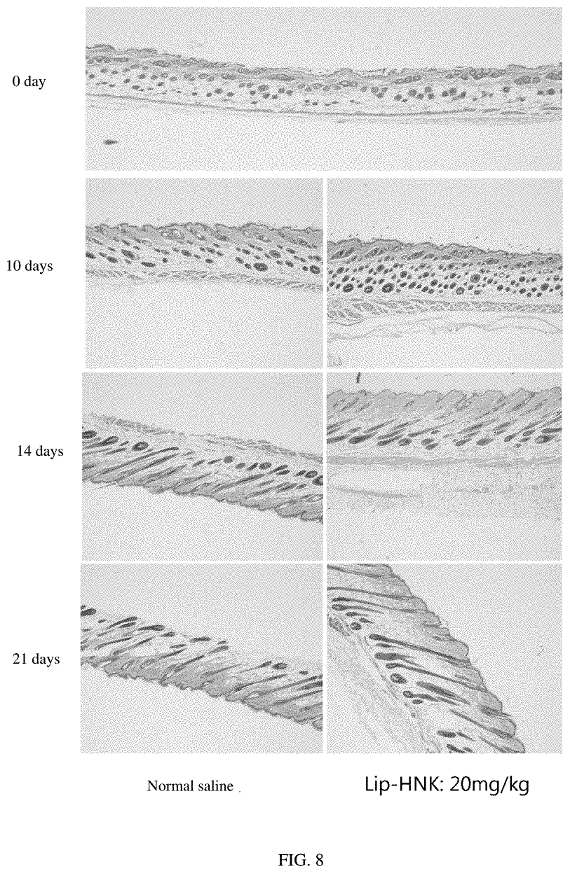

FIG. 8 shows that the follicle growth period in the honokiol liposome group was earlier than that in normal saline control group, and the number of hair follicles was significantly increased compared with the normal saline control group. Moreover, Table 3 shows that the hair follicle length in the honokiol liposome group was greater than that in normal saline control group.

TABLE 3
Analysis of Hair Follicle Length in Mice at Different Treatment Times

| | Hair follicle length (μm) | | | |
|---|---|---|---|---|
| Groups | 0 day | 10 days | 14 days | 21 days |
| Normal | 21.89 ± 4.06 | 28.61 ± 6.48 | 89.95 ± 6.98 | 149.17 ± 17.72 |
| saline | | | | |
| control | | | | |
| group | | | | |
| Honokiol | 21.89 ± 4.06 | 63.99 ± 11.08 | 134.43 ± 53.99 | 251.56 ± 13.55 |
| liposome | | | | |
| group | | | | |

3) Hepatorenal Toxicity

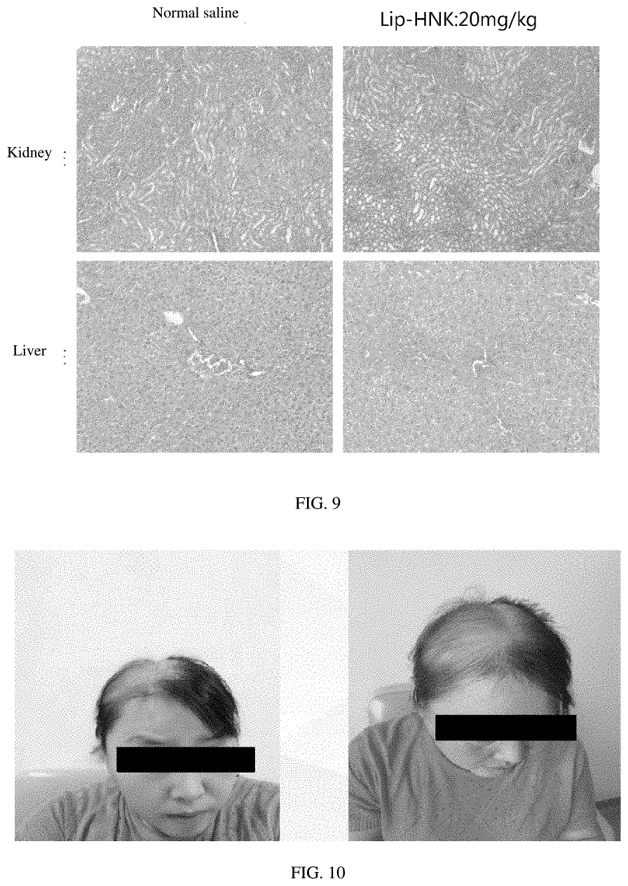

To further clarify whether honokiol liposome has toxic effects while promoting hair growth, liver and kidney tissues were taken for paraffin embedding and stained with HE (HE staining procedure for liver and kidney tissue is the same as HE staining procedure for skin tissue). FIG. 9 shows that there was no significant hepatorenal toxicity after intraperitoneal injection of honokiol liposome, and there was no death and no effect on body weight in mice compared to normal saline control group.

In addition, in clinical studies of honokiol liposome for the treatment of brain gliomas, the inventor found that honokiol can promote hair growth as well as white hair blackening. FIG. 10 shows hair growth increased and white hair blackening in patients after intravenous injection of honokiol liposome.

1. A method of inhibiting medulloblastoma comprising administering honokiol.

2. The method of claim 1, wherein the honokiol is prepared as honokiol liposome.

3. The method of claim 1, wherein the honokiol inhibits proliferation of medulloblastoma cells.

4. The method of claim 1, wherein the honokiol induces apoptosis of medulloblastoma cells.

5. The method of claim 4, wherein the honokiol induces the apoptosis of the medulloblastoma cells through ROS/ERK/p38MAPK pathway.

6. The method of claim 4, wherein the honokiol induces the apoptosis of the medulloblastoma cells through a Caspase-dependent pathway.

7. The method of claim 1, wherein the honokiol induces cell cycle arrest of medulloblastoma.

8. A method of promoting hair growth comprising administering honokiol.

9. The method of claim 8, wherein the hair growth occurs at head.

10. A method of promoting white hair blackening comprising administering honokiol.

11. The method of claim 2, wherein the honokiol liposome is administered through injection.

12. The method of claim 1, wherein the honokiol induces G1 cycle arrest of medulloblastoma.
